(19) United States
(12) Patent Application Publication
(10) Pub. No.: US 2008/0082168 A1
(43) Pub. Date: Apr. 3, 2008

(54) SURGICAL SCAFFOLD TO ENHANCE FIBROUS TISSUE RESPONSE

(76) Inventors: Marc Peterman, Austin, TX (US); David Hooper, Austin, TX (US); Abhijeet Joshi, Austin, TX (US); Peter Tarcha, Lake Villa, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/831,802

(22) Filed: Jul. 31, 2007

Related U.S. Application Data
(63) Continuation-in-part of application No. 11/541,356, filed on Sep. 29, 2006.

(51) Int. Cl. A61F 2/44

(52) U.S. Cl. 623/17.11

(57) ABSTRACT

Some embodiments of the present disclosure relate to an implantable device for the closure and/or repair of a spinal defect (e.g., posterior annular defects) and/or preventing recurring herniation. An implantable medical device, in some embodiments, may be coated or impregnated with a releasable pharmaceutical compound. Accordingly, some embodiments of the present disclosure relate to compositions that include one or more pharmaceutical compounds. In addition, some embodiments of the disclosure relate to methods for making and using compositions and medical implants. A spinal implant may include, for example, a scaffold having a substantially planar surface; and a plurality of tails, each of which has a first end that is attached to the scaffold and a second end that is configured and arranged to be threaded through a respective perforation in a vertical body, wherein the scaffold comprises a pharmaceutically effective amount of a pharmaceutical agent.

### CROSS-REFERENCE TO RELATED APPLICATIONS

This application is a continuation-in-part of U.S. patent application Ser. No. 11/541,356 filed Sep. 29, 2006, the contents of which are incorporated herein in their entirety by reference.

### BACKGROUND

The spine comprises vertebrae and intervertebral discs separating the vertebrae. Intervertebral discs comprise a tough, fibrous outer ring, called the annulus fibrosis, and a viscous, fluid-filled central core called the nucleus pulposus. The annulus fibers are attached to vertebral end plates (cartilage) in the inner portion and peripherally, they are attached directly to the vertebral bone. Thus, the nucleus pulposus is contained in a compartment defined by the annulus fibrosis, vertebral end plates and the adjoining vertebrae.

A healthy spine owes much of its flexibility and range of motion to the ability of intervertebral discs to deform (e.g., compress) and recover in response to the application and release of a deforming force (strain). This capability may be compromised, however, by any damage or condition (e.g., degenerative bone disease) that weakens the annulus fibrosis due to abnormal loading and/or results in extravasation of nucleus pulposus. In addition, if the annulus fibrosis is damaged (egg, herniated disc) such that the annulus fibrosis and/or nucleus pulposus contact a nerve (e.g., a nerve root and/or the spinal cord), a subject may experience substantial back and/or leg pain.

Prior to the instant disclosure, options for intervention to treat such spinal conditions have been limited. For example, the large intradiscal pressures generated during normal loading of the spine may interfere with normal healing processes. Sutures in the annulus fibrosis may pull out quickly, may aggravate existing tears and/or may nucleate new tears. Implantable patches attached to the annulus fibrosis may have the same adverse results. In addition, since most of the annulus fibrosis ordinarily lacks a direct blood supply, options for delivering locally-acting pharmaceuticals have been limited.

### SUMMARY

Therefore, a need exists for implants, compositions, and methods for the repair of annular defects. Some embodiments of the present disclosure relate to an implantable device for the closure and/or repair of a spinal defect (e.g., posterior annular defects) and/or preventing recurring herniation. An implantable medical device, in some embodiments, may be coated or impregnated with a releasable pharmaceutical compound. Accordingly, some embodiments of the present disclosure relate to compositions that include one or more pharmaceutical compounds. In addition, some embodiments of the disclosure relate to methods for making and using compositions and medical implants.

A spinal implant may include, for example, a scaffold having a substantially planar surface; and a plurality of tails, each of which has a first end that is attached to the scaffold and a second end that is configured and arranged to be threaded through a respective perforation in a vertical body, wherein the scaffold comprises a pharmaceutically effective amount of a pharmaceutical agent.

According to some embodiments, a spinal implant may include an implantable obturator configured and arranged to cover an annular defect, wherein the implantable obturator comprises a pharmaceutical agent; and a plurality of tails, each of which has a first end that is attached to the implantable obturator and a second end that is configured and arranged to be threaded through a respective perforation in a vertical body. An implantable obturator may be contoured, for example, to cover the annular defect and comprises a resilient or rigid material. An annular defect, in some embodiments, to be covered may be on an anterior portion of a disc, on a posterior portion of a disc, and/or on a lateral portion of a disc.

A pharmaceutical agent may be included in a pharmaceutical agent elution matrix that is configured and arranged to release the pharmaceutical agent upon implantation. A spinal implant may further include a coating on at least a potion of the spinal implant, the coating comprising the pharmaceutical agent elution matrix. A scaffold and/or an implantable obturator, in some embodiments, may include a first surface and a second surface. A first surface may be configured and arranged to face the annulus and nucleus pulposus upon implantation and may comprise a pharmaceutical agent elution matrix. In some embodiments, a scaffold and/or an implantable obturator may include a biocompatible material and/or a biodegradable material. A spinal implant may include polyester, polytetrafluoroethylene, or polyester and/or polytetrafluoroethylene in some embodiments. A spinal implant may include a polymer selected from the group consisting of a phosphorylcholine linked macromolecule, an oligoethylenimine, and a polyethylenimine.

A spinal implant, according to some embodiments, may further include a first tail configured and arranged to be threaded through a first perforation in a first vertical body and a second tail configured and arranged to be threaded through a first perforation in a second vertical body. In some embodiments, a spinal implant in some embodiments, may further include a third tail configured and arranged to be threaded through a second perforation in a first vertical body and a fourth tail configured and arranged to be threaded through a second perforation in a second vertical body.

A pharmaceutical agent, in some embodiments, may be selected from the group consisting of an analgesic, an antimicrobial agent, an anti-inflammatory agent, a fibrosis-inducing agent (e.g., an adhesive, an arterial vessel wall irritants a bone morphogenic protein, an extracellular matrix component, an inflammatory cytokine, a polymer, and combinations thereof), and combinations thereof. A fibrosis-inducing agent may be selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

According to some embodiments, a spinal implant may include a scaffold and/or an implantable obturator having a regular curvilinear shape (e.g., an oval, a rectangle, a square, and an ellipse). A scaffold and/or an implantable obturator may be from about 2 mm to about 30 mm along its longest axis, from about 2 mm to about 30 mm along its shortest axis, and/or from about 1 um to about 10 mm at its point of maximum thickness.

A system for implanting a spinal implant, in some embodiments, may include a spinal implant comprising a scaffold having a releasable pharmaceutical agent and a plurality of tails, wherein each tail is configured and arranged to be threaded through a respective perforation in a vertical body; and an apparatus for placing the spinal implant in or along the spine comprising: a first handle having a channel, a body having a channel, a hollow shaft or tube that connects the channel of the first handle to the channel of the body to form an inserter track, an elongate inserter slidably contained in the inserter track, wherein the inserter has a body end proximal to the body and a first handle end proximal to the first handle, and wherein the body end comprises an opening configured and arranged to receive at least one of the plurality of tails, a second handle attached to the inserter at its first handle end and operable to slide the inserter back and forth along the inserter track, and a pair of articulating needles or guides configured and arranged to contact at least one of the plurality of tails and thread it through the respective perforation in a vertebral body. A system for implanting a spinal implant, in some embodiments, may be configured and arranged to be disposable.

The present disclosure also relates to a method of obturating an annular defect, in some embodiments, said method comprising contacting the annular defect with a spinal implant comprising (a) an implantable obturator configured and arranged to cover an annular defect, wherein the implantable obturator comprises a pharmaceutical agent; and (b) a plurality of tails, each of which has a first end that is attached to the implantable obturator and a second end that is configured and arranged to be threaded through a respective perforation in a vertical body.

The disclosure also relates, according to some embodiments, to methods of manufacturing a spinal implant. For example, a method may include providing a spinal implant comprising an implantable obturator configured and arranged to cover an annular defect and having a first surface and a second surface, and a plurality of tails, each of which has a first end that is attached to the implantable obturator and a second end that is configured and arranged to be threaded through a respective perforation in a vertical body, coating the first surface with a pharmaceutical agent elution matrix comprising a pharmaceutical agent; and sterilizing the spinal implant.

A method of inducing fibrosis at or near an annular defect may include, for example, contacting the annular defect with a spinal implant comprising (a) an implantable obturator configured and arranged to cover an annular defect, wherein the implantable obturator comprises a pharmaceutical agent; and (b) a plurality of tails, each of which has a first end that is attached to the implantable obturator and a second end that is configured and arranged to be threaded through a respective perforation in a vertical body. In some embodiments, a method of inducing fibrosis at or near an annular defect may also include irradiating the annular defect.

### BRIEF DESCRIPTION OF THE DRAWINGS

Some embodiments of the disclosure may be understood by referring, in part, to the following description and the accompanying drawings, wherein:

FIG. 1 shows a herniated disc, suitable for repair by a scaffold implant of the disclosure;

FIG. 2 illustrates another view of a herniated disc, suitable for repair by a scaffold implant of the disclosure;

FIG. 3 depicts a scaffold according to an embodiment of the disclosure used to repair a disc defect;

FIG. 4 shows details of making a pair of perforations in vertebral body endplates according to an embodiment of the disclosure;

FIG. 5 illustrates peri-annular placement of a scaffold to repair an annular defect according to an embodiment of the disclosure;

FIG. 6 depicts intra-annular placement of a scaffold to repair an annular defect according to an embodiment of the disclosure;

FIG. 7 shows details of a scaffold implant according to an illustrative embodiment of the disclosure;

FIG. 8 illustrates one technique for tying a knot in a scaffold implant according to an embodiment of the disclosure;

FIG. 9 depicts one part of a manual technique for threading the tails of a scaffold implant into respective perforations or openings in adjoining spine's vertebral bodies according to an embodiment of the disclosure;

FIG. 10 shows another part of a manual technique for threading the tails of the scaffold implant into the respective perforations or openings in the spine's vertebral bodies according to one illustrative embodiment of the disclosure;

FIG. 11 illustrates an instrument for threading the tails of the scaffold implant into the respective perforations or openings in the spine's vertebral bodies according to one illustrative embodiment of the disclosure; and

FIG. 12 depicts details of the operation of the instrument shown in FIG. 11.

### DETAILED DESCRIPTION

Implanting a medical device in a subject's body may be correlated with an inflammatory and/or cytotoxic response. For example, a subject's body may produce fibrotic tissue that partially or completely encapsulates the foreign body. Such responses may be regarded as undesirable under some circumstances. For example, a subject in whom fibrotic tissue contacts a nerve following spinal surgery may experience significant back and/or leg pain. According to some embodiments of the present disclosure, however, these responses may be harnessed and/or enhanced for beneficial and/or desirable purposes. For example, formation of fibrotic tissue around an annular implant may repair an annular defect alone or in combination with the implant.

The present disclosure relates to apparatus, compositions, systems, and methods for treating a spinal condition (e.g., an annular defect). A variety of pathologic conditions may yield herniated nucleus pulposus, such as acute traumatic tears or cumulative delamination of the annular fibers. Cumulative damage may result from dehydration of the nucleus pulposus, which may change the loading environment of the posterior annulus. Thus, this desiccation may contribute to mechanical failure of the structure. Extrusion of the nucleus pulposus may occur if the annulus is compromised. Patients with radiographic evidence of tears and associated herniations may be asymptomatic. Radiculitis, however, may be an indication for surgical intervention, where the neuropathic symptom is secondary to mechanical impingement and autoimmune response to nucleus material. Structural changes in the posterior portion of the anterior column also may produce neovascularization and/or nociceptive changes, which may contribute to axiomechanical back pain. Surgeons frequently operate on leg pain or radiculopathy over axiomechanical back pain because the probability of success may be higher and long-term consequences of untreated neural compression exist. Discectomy may be the most common intervention for radiculopathy, wherein the offending fragment is removed. Regardless of the source, the pathology ultimately results from mechanical deficiency of the posterior annulus fibrosis. The extruded fragment may be surgically removed without addressing the annular defect, mechanical change, or inflammation.

A common zone for herniation may be in the posterolateral region. The posterior annulus may be relatively thin. The central region is reinforced by the posterior longitudinal ligament (PLL), thus discs may herniate posteriorly and lateral to the PLL. Anterior or direct lateral herniation may be rare.

### Apparatus

An apparatus, according to some embodiments, may include a spinal implant (e.g., a scaffold implant) or a device for placing an implant (e.g., a scaffold implant) in a spine. For example, an apparatus may include a spinal implant configured and arranged for placement on, near, and/or adjacent to an annular defect. In some embodiments, an implant may include a scaffold (e.g., mesh and/or patch) having at least one tail configured and arranged to contact a vertebral body of the spine. For example, an implant may include one tail, two tails, three tails, four tails, or more than four tails. Tails may be spaced apart on an implant at desired intervals, regular intervals, and/or irregular intervals, according to some embodiments. A tail and a scaffold, in some embodiments, may be made from the same materials or different materials. In some embodiments where a tail and a scaffold have different compositions, a tail may include a wire.

An apparatus, according to some embodiments of the disclosure, may be sized according to the intended application. For example, the length of the tail(s), the size and shape of the scaffold, and the site of attachment between the tail(s) and the scaffold may be selected to accommodate the spine of the intended subject, whether a child or an adult, whether its morphology is regular or unusual. In some embodiments, an apparatus may include a scaffold having a generally oval shape (or a generally rectangular shape) and a total of two tails, one attached at either end of the scaffold. This scaffold may measure from about two millimeters (2 mm) to about thirty millimeters (30 mm) along its longest axis, from about two millimeters (2 mm) to about thirty millimeters (30 mm) along its shortest axis, and/or from about one micron (1 μm) to about ten millimeters (10 mm) at its point of maximum thickness. A scaffold may measure from about 2 mm to about 6 mm, from about 2 mm to about 8 mm, from about 2 mm to about 10 mm, from about 4 mm to about 10 mm, from about 4 mm to about 12 mm, from about 4 mm to about 18 mm, from about 4 mm to about 24 mm, and/or from about 6 mm to about 24 mm along its longest axis.

Tails may be sized to include a generous excess after being secured to a vertebral body. This excess may be trimmed as desired. A tail may be from about one centimeter (1 cm) to about fifteen centimeters (15 cm). In some embodiments, an apparatus may include a scaffold having a generally rectangular shape (or a generally oval shape) and a total of four tails, one attached at each corner. Where a scaffold includes more than one tail, the tails may be sized independently or identically as desired or required by the particular intended application.

According to some embodiments, an implant may include an annular scaffold (e.g., mesh and/or patch) that may be applied to a nucleus pulposus or nucleus of a disc in a spine. An implant may be secured to itself (e.g., the implant tails may be tied to the scaffold), rather than using rigid fasteners, such as screws, plugs, etc. in some embodiments.

As persons of ordinary skill in the art understand, a herniated disc may result in release of nucleus matter. A device, according to some embodiments, may partially or completely retain and/or contain herniated nucleus pulposus and/or prevent herniation (egg, an implantable obturator), thus avoiding potential contact to peripheral nerve roots. In some embodiments, a device may also support reintroduction of extruded nucleus pulposus materials into the disc space. A device may further retain and/or contain another device (e.g., a nucleus replacement implant). In contrast, an apparatus according to some embodiments may not form a barrier and may not itself contain herniated nucleus pulposus or obturate a tear in the annulus fibrosis. For example, an apparatus may serve as a scaffold for formation of new tissue (e.g., scar tissue) such that the new tissue repairs the defect. In some embodiments, a spinal implant may function to contain and/or prevent herniation for an initial period and subsequently biodegrade (e.g., once fibrous tissue has grown in).

According to some embodiments, a scaffold may include pliable materials (e.g., mesh, fabric, felt) and lack a permanent structure (a “pliable scaffold”). A scaffold, in some embodiments, may include rigid and/or semi-rigid materials such that it retains or at least tends to retain its shape (a “rigid scaffold”), A rigid scaffold may be configured and arranged (either beforehand or in situ) to conform to the contours of the spine where it is to be placed. In addition, a rigid scaffold may be configured and arranged to nucleate and/or support formation of fibrotic tissue that conforms to the contours of the spine where the implant is to be placed and/or seals an annular defect. In some embodiments, a scaffold may be laterally reinforced by including, for example, a woven material having a more rigid weave in at least one dimension, a secondary element (e.g., a plastic insert), and/or a wire (e.g., a shape memory alloy).

An implant may include a permeable and/or an impermeable scaffold, according to some embodiments. A scaffold may include, in some embodiments, a releasable pharmaceutical agent and a polymer. A scaffold may be configured and arranged to be degradable (e.g., biodegradable) and/or non-degradable, A scaffold may include a pharmaceutical agent elution matrix configured and arranged to permit sustained, graduated, and/or, periodic release of a pharmaceutical agent. In some embodiments, surface characteristics of a scaffold material may be prepared and/or modified (e.g., by texturing) to support release of a pharmaceutical agent. For example, a scaffold may be nanotextured with tubules. A scaffold may be coated with a pharmaceutical agent in a suitable carrier configured and arranged to have desired release capabilities.

If desired or necessary, a pharmaceutical agent may include a binder to carry, load, of allow sustained release of the agent, such as but not limited to a suitable polymer or similar carrier. According to some embodiments of the disclosure, a polymer may include a product of a polymerization reaction inclusive of homopolymers, copolymers, terpolymers, etc., whether natural or synthetic, including random, alternating, block, graft, branched, cross-linked, blends, compositions of blends and variations thereof. A polymer may be in true solution, saturated, or suspended as particles or supersaturated in the therapeutic agent. A polymer may be biocompatible and/or biodegradable.

A polymeric material may include a phosphorylcholine linked macromolecule in some embodiments (a “PC polymer”). For example, a polymeric material may include a macromolecule containing pendant phosphorylcholine groups such as poly(MPC_{w}:LMAₓ:HPMA_{y}:TSMA_{z}), where MPC is 2-methacryoyloxyethylphosphorylcholine, LMA is lauryl methacrylate, HPMA is hydroxypropyl methacrylate and TSMA is trimethoxysilylpropyl methacrylate, and w, x, y, and z are molar ratios of the monomers used in the feed. These values may be 23, 47, 25, and 5, respectively, but they are not necessarily the ratios that exist in the finished polymer.

A PC polymer may include, for example, 5% pendant trimethoxysilane groups, which may be used to crosslink the polymer after it is coated on a surface. These groups may also be used to chemically bond the material to a device having an appropriate surface chemistry. For example, where a scaffold that includes a Dacron mesh, the surface of the polyester may be hydrolyzed producing hydroxyl groups for reaction with trimethoxy silane. Alternatively, the Dacron may be formulated with impregnated fiber glass or glass powder. The glass may be the source of surface hydroxyl groups; however, it may change the mechanical properties of the Dacron.

A scaffold may include a polymer selected from the group consisting of alginate, aliphatic polyesters, bioglass, blood cells, bone-allograft or autograft, bone cement, carbohydrates, cellulose, cellulose derivatives (e.g., HPC), ceramics, chitin, chitosan, chitosan derivatives, collagen, collagen—native fibrous, collagen—recombinant derived, collagen—reconstituted fibrous, collagen—soluble, collagen—Types 1 to 20, copolymers of glycolide, copolymers of lactide, cyanoacrylate, dacron, demineralized bone, elastin, felt, fibrin, gelatin, glass, glycolide/1-lactide copolymers (PGA/PLLA), glycolide/trimethylene carbonate copolymers (PGA/TMC), glycosaminoglycans, gold, hyaluronic Acid, hyaluronic acid derivatives, hydrogel, hydroxy apatite, hydroxyethyl methacrylate, lactide/ε-capiolactone copolymers, lactide/σ-valerolactone copolymers, lactide/tetramethylglycolide copolymers, lactide/trimethlylene carbonate copolymers, 1-lactide/dl-lactide copolymers, polymethyl methacrylate (PMMA), polymethyl methacrylate-N-vinyl pyrrolidone copolymers, polymethyl methacrylate-styrene (MMA-styrene), nitinol, nylon-2, oligoethylenimine (OEI), OEI-HD (e.g., a condensation product of OEI with hexanedioldiacrylate), oxidized regenerated cellulose, PHBA/γ-hydroxyvalerate copolymers (PUBA/UVA), phosphate glasses, PLA/polyethylene oxide copolymers, PLA-polyethylene oxide (PELA), polyethylenimine (PEI), poly (amino acids), poly (trimethylene carbonates), poly hydroxyalkanoate polymers (PHA), poly(alkylene oxalates), poly(butylene diglycolate), poly(glycerol sebacate), poly(hydroxy butyrate) (PHB), poly(methacrylic acid), poly(n-vinyl pyrrolidone), poly(ortho esters), poly(styrene sulfonate), poly-β-alkanoic acids, poly-β-hydroxybutyrate (PBA), poly-β-hydroxypropionate (PHPA), poly-β-malic acid (PMLA), poly-ε-caprolactone (PCL), poly-σ-valerolactone, polyalkyl-2-cyanoacrylates, polyanhydrides, polycyanoacrylates, polydepsipeptides, polydihydropyrans, poly-DL-lactide (PDLLA), polyester, polyesteramides, polyester-polyallylene oxide block copolymers, polyesters of oxalic acid, polyethylene glycol—crosslinked, polyethylene glycol—poly(vinyl PEG), polyethylene glycol (PEG), polyethylene oxide, polyglycan esters, polyglycolide (PGA), polyiminocarbonates, polylactides (PLA), poly-1-lactide (PLLA), polymethyl methacrylate (PMMA), polyorthoesters, poly-p-dioxanone (PDO), polypeptides, polyphosphazenes, polysaccharides, polyurethanes (PU), polyvinyl alcohol (PVA), pseudo-poly(amino acids), radiopacifiers, salts, silicone, silk, starch, steel (e.g. stainless steel), synthetic cancellous bone void fillers, synthetic polymers, titanium, tricalcium phosphate, tyrosine based polymers, and combinations thereof. A scaffold may include a material selected from the group consisting of bone chips, calcium, calcium carbonate, calcium phosphate, calcium sulfate, liposomes, mesenchymal cells, osteoblasts, platelets, proteins (e.g., albumin, casein, whey proteins, plant proteins, and fish proteins), proteins modified, thrombin, trimethylene carbonate (TMC), and combinations thereof.

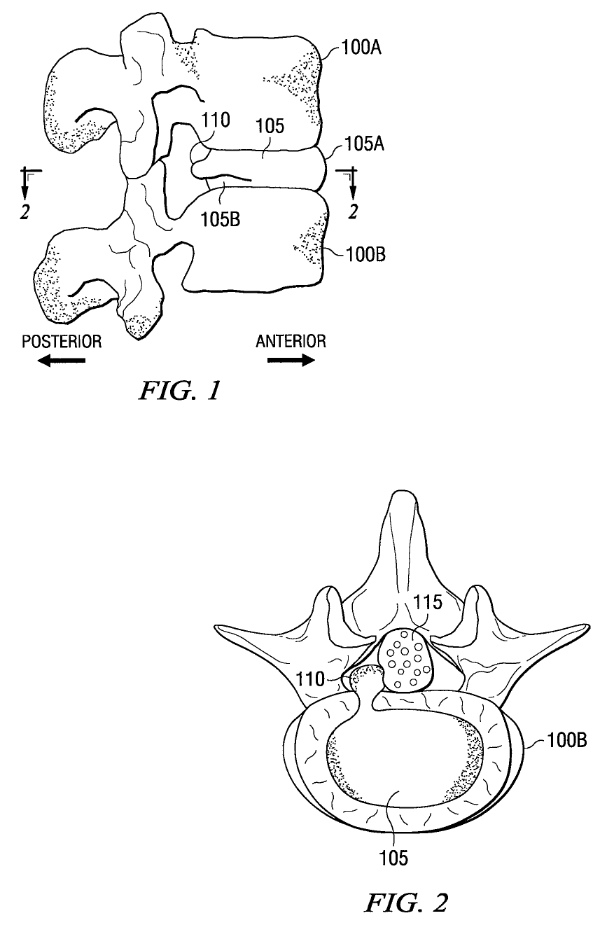

FIG. 1 shows a herniated disc, suitable for repair by a disclosed implant. Nucleus 105 resides between vertebral body 100A and vertebral body 100B. Nucleus 105 includes anterior annulus 105A and posterior annulus 105B. A posterior annular tear may result in release of the nucleus pulposus, thus producing a bulge 110 and possibly release of the nucleus pulposus, otherwise known as a herniated disc.

FIG. 2 illustrates another view of a herniated disc, suitable for repair by a disclosed implant. More specifically, FIG. 2 illustrates parts of the structures in FIG. 1, when sliced or viewed along line A-A, i.e., a top or transverse view.

When viewed from the top, one may observe that bulge 110 may come in contact with, or exert pressure to surrounding structures or tissues. For example, bulge 110 may compress neural element 115. As a result, the patient may experience pain, discomfort, or loss of function. In the case of a tear, the leakage of nucleus pulposus may result in a variety of problems and complications, as persons of ordinary skill in the art understand.

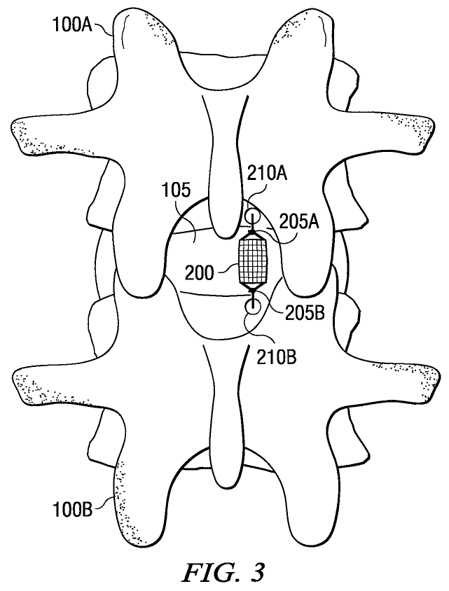

One may repair a disc defect (e.g., annular tear) by applying an embodiment of a scaffold implant of the disclosure FIG. 3 depicts a scaffold implant according to an embodiment of the disclosure used to repair a disc defect. The implant includes scaffold 200, secured to vertebral body 100A and to vertebral body 100B.

Scaffold 200 attaches to vertebral body 100A through perforation 210A, and to vertebral body 100B through perforation 210B. The respective positions of perforation 210A and perforation 210B depend on a number of factors, including the desired placement of scaffold 200.

A practitioner (erg, a surgeon) may position scaffold 200 in a defective or damaged area of the disc, e.g., over an annular tear. In one embodiment, perforation 210A and perforation 210B reside in the posterior margins of vertebral body 100A and vertebral body 100B, respectively. In other embodiments, one may select the precise positions of perforation 210A and perforation 210B depending on factors such as the desired position of scaffold 200, a patient's anatomy, the nature of the defect in the disc, etc., as persons of ordinary skill in the art who have the benefit of the instant disclosure understand

### Compositions

An implant, according to some embodiments, may include a composition to elicit a specific biologic response. In some embodiments, an implant may include a composition formulated to enhance annular defect repair (e.g., by augmenting and/or inhibiting one or more biological processes) according to some embodiments. For example, an implant may include a releasable pharmaceutical agent that enhances or impedes fibrosis, A pharmaceutical agent may include, for example, an anti-inflammatory agent, an anti-adhesive agent, and/or a pro-adhesive agent.

In some embodiments, a pharmaceutical agent may result in adhesion and/or fibrosis in one or more surrounding tissues. Production of fibrotic tissue at or near the site of a disc defect may enhance defect repair and/or treatment. For example, new fibrotic tissue that partially and/or completely surrounds an implant or defect may at least partially contain the nucleus pulposus, thereby augmenting the native annular fibrosis. A scaffold, in some embodiments, may include an anti-adhesion compound (ergo, on a portion of the scaffold that may contact a nerve root to minimize or avoid painful tethering of scar tissue to a nerve root).

According to some embodiments, a composition including a pharmaceutical agent may be carried on and/or eluted by at least a portion of an implant. Thus, a scaffold may have one or more portions that include a therapeutic composition and one or more portions that lack a therapeutic composition. For example, a scaffold may have a domain or domains configured and arranged to confer structure (e.g., shape, rigidity, resilience, etch) and a domain or domains containing a pharmaceutical agent. In a non-limiting example, a scaffold may include opposing surfaces, one of which includes biocompatible polymers for structure and the other of which includes a pharmaceutical agent. One of ordinary skill in the art having the benefit of the present disclosure will understand that determining which surface faces the annular defect and which surface faces away from the annular defect will depend, at least in part on what pharmaceutical agent(s) are used, the shape of the scaffold, the nature of the adjoining tissue. In another non-limiting example, a scaffold may include a sheath of a biocompatible polymer for structure and a core comprising a bioactive agent. The sheath may be configured and arranged to be biodegradable and/or bioresorbable (e.g., to permit the scaffold to function as a barrier for an initial period).

A pharmaceutical agent suitable for inclusion in a scaffold of the disclosure, in some embodiments, may include a protein (e.g., peptide (e.g., adhesion peptide), enzyme, antibody, receptor, receptor ligand), a carbohydrate (e.g., monosaccharide, disaccharide, polysaccharide (linear or branched)), a lipid (e.g., prostaglandin, eicosanoid, steroid), a nucleic acid (e.g., DNA, RNA, siRNA, microRNA, ribozyme, virus, vector, coding sequence, antisense sequence, nucleotide), and/or combinations thereof. In some embodiments, a pharmaceutical agent may include one or more of the compounds listed in TABLE 1 and/or analogues and derivatives thereof. For example, a pharmaceutical agent may include alpha-interferon, an amino acid, an angiogenic agent, an anti-allergic agent, an anti-angiogenic agent, an antiarrhythmic agent, an antibiotic, an anti-coagulant agent, an anti-fibrin agent, an anti-fungal agent, an anti-inflammatory agent, an anti-neoplastic agent, an antioxidant, an anti-platelet agent, an anti-proliferative agent, an anti-rejection agent, an anti-thrombotic agent, an anti-viral drug, bioactive RGD, a blood clotting agent, a cell, a chemotherapeutic agent, a fibrosis-inducing agent, a fibrosis-inhibiting agent, a growth factor, a hormone, a nitric oxide or a nitric oxide donor, nitroprusside, a phosphodiesterase inhibitor, a proliferative agent, a prostaglandin inhibitor, a proteoglycan, a radioactive material, a serotonin blocker, a super oxide dismutase, a super oxide dismutase mimetic, suramin, a thioprotease inhibitor, thiazolopyrimidine, a tyrosine kinase inhibitor, a vasodilator, and/or a vitamin. In some embodiments, a pharmaceutical agent may include a compound selected from the group consisting of 1-α-25 dihydroxyvitamin D₃, alcohol, all-trans retinoic acid (ATRA), angiotensin II antagonists, anti-tumor necrosis factor, beta-blocker, carcinogens, chondroitin, clopidegrel, collagen inhibitors, colony stimulating factors, coumadin, cyclosporine A, cytokines, dentin, diethylstibesterol, etretinate, glucosamine, glycosaminoglycans, growth factor antagonists or inhibitors, heparin sulfate proteoglycan, immoxidal, immune modulator agents (e.g., immunosuppressant agents), inflammatory mediator, insulin, isotretinoin (13-cis retinoic acid), lipid lowering agents (e.g., cholesterol reducers, HMC-CoA reductase inhibitors (statins)), lysine (e.g., polylysine), methylation inhibitors, N[G]-nitro-L-arginine methyl ester (L-NAME), plavix, polyphenol, PR39, prednisone, signal transduction factors, signaling proteins, somatomedins, thrombin, thrombin inhibitor, ticlid, and combinations thereof.

TABLE 1
Pharmaceutical Agents

```
alpha-interferon
amino acid
  L-arginine
analgesic
  acetaminophen
  aspirin
  codeine
  cox2 inhibitor
  ibuprofen
  morphine
  naproxin
  nonsteroidal anti-inflammatory drug
angiogenic agent
  angiogenin
  angiotropin
  bone morphogenic protein (BMP)
  epidermal growth factor (EGF)
  fibrin
  fibroblast growth factor - acidic (aFGF) and basic (bFGF)
  granulocyte-macrophage colony stimulating factor (GM-CSF)
  hepatocyte growth factor (HGF)
  hypoxia-inducible factor-1 (HIF-1)
  indian hedgehog (inh)
  insulin growth factor-1 (IGF-1)
  interleukin-8 (IL-8)
  macrophage antigen 1 (Mac-1)
  nicotinamide
  platelet-derived endothelial cell growth factor (PD-ECGF)
  platelet-derived growth factor (PDGF)
  transforming growth factors α (TGF-α) & β (TGF-β)
  tumor necrosis factor-α (TNF-α)
  vascular endothelial growth factor (VEGF)
  vascular permeability factor (VPF)
anti-allergic agent
  permirolast potassium
antiarrhythmic agent
  amiodarone
  diltiazem
  lidocaine
  procainamide
  sotalol
antibiotic
  cipro
  erythromycin
  flagyl
  imipenem
  penicillin
  vancomycin
  zosyn
anti-coagulant agent
  heparin
  lovenox
anti-fibrin agent
anti-fungal agent
anti-inflammatory agent
  aspirin
  clobetasol
  colchicine
  dexamethasone
  glucocorticoid
    betamethasone
    budesonide
    cortisone
    dexamethasone
    hydrocortisone
    methylprednisolone
    prednisolone
  non-steroidal anti-inflammatory agent
    acetominophen
    diclofenac
    diclofenac
    diflunisal
    etodolac
    fenoprofen
    flurbiprofen
    ibuprofen
    indomethacin
    ketoprofen
    ketorolac
    meclofenamic acid
    naproxen
    phenylbutazone
    piroxicam
    sulindac
  tacrolimus
anti-neoplastic agent
  alkylating agent
    altretamine
    bendamucine
    carboplatin
    carmustine
    cisplatin
    cyclophosphamide
    fotemustine
    ifosfamide
    lomustine
    nimustine
    prednimustine
    treosulfin
  antibiotic
    doxorubicin hydrochloride
    mitomycin
  antimetabolite
    azathioprine
    fluorouracil
    gemcitabine
    mercaptopurine
    methotrexate
    pentostatin
    trimetrexate
  antimitotic agent
    docetaxel
    paclitaxel
    vinblastine
    vincristine
  ceramide
  estradiol (e.g., 17-β-estradiol)
  flutamide
  imatinib
  levamisole
  oxaliplatin
  tamoxifen
  taxol
  topotecan
antioxidant agent
anti-platelet agent
  eptifibatide
  forskolin
  GP IIb/IIIa inhibitor
    L-703,081
anti-proliferative agent
  (+)-trans-4-(1-aminoethyl)-1-(4-pyridylcarbamoyl) cyclohexane
  amlodipine
  angiotensin converting enzyme inhibitor
    captopril
    cilazapril
    lisinopril
  anti-estrogen
    tamoxifen
  anti-restenosis agent
    40-O-(2-hydroxyethyl)rapamycin (everolimus)
    40-O-(2-hydroxyethyoxy)ethylrapamycin
    40-O-(3-hydroxypropyl)rapamycin
    40-O-tetrazolylrapamycin (zotarolimus, ABT-578)
    adenosine A2A receptor agonist
    pimecrolimus
    rapamycin (sirolimus)
    rapamycin analog
    tacrolimus
  azathioprine
  benidipine
  calcium channel blocker
    nifedipine
  cilnidipine
  cytostatic agent
    angiopeptin
  diltiazem and verapamil
  docetaxel
  doxorubicin hydrochloride
  fibroblast growth factor antagonists
  fish oil (omega 3-fatty acid)
  fluorouracil
  histamine antagonist
  lercanidipine
  lovastatin
  methotrexate
  mitomycin
  paclitaxel
  rho kinase inhibitor
  trifluperazine
  topoisomerase inhibitor
    etoposide
    topotecan
  vinblastine
  vincristine
anti-rejection agent
anti-thrombonic agent
  argatroban
  dextran
  dipyridamole
  D-phe-pro-arg-chloromethylketone (synthetic antithrombin)
  bivalirudin
  fondaparinux
  forskolin
  GP IIb/IIIa inhibitor
    L-703,081
    glycoprotein IIb/IIIa platelet membrane receptor antagonist antibody
  heparinoid
  hirudin
  low molecular weight heparin
  prostacyclin
  prostacyclin analogue
  recombinant hirudin
  sodium heparin
  thrombolytics
    urokinase
    recombinant urokinase
    pro-urokinase
    tissue plasminogen activator
    tenecteplase (TNK-tPA)
  vapiprost
anti-viral drug
bioactive RGD
blood clotting agent
  streptokinase
  tissue plasminogen activator
cell
  bacteria
  blood cell
  bone marrow
  fat cell
  genetically engineered epithelial cell
  lymphocytes
  muscle cell
  stem cell
  umbilical cord cell
  yeast
    Ziyphi fructus
fibrosis-inducing agent
  adhesive
    crosslinked poly(ethylene glycol)-methylated collagen
    cyanoacrylate
  arterial vessel wall irritant
    crystalline silicates
    copper
    ethanol
    metallic beryllium and oxides thereof
    neomycin
    quartz dust
    silica
    silk
    talc
    talcum powder
    wool
  bleomycin
  bone morphogenic protein (BMP)
    bone morphogenic protein-2
    bone morphogenic protein-3
    bone morphogenic protein-4
    bone morphogenic protein-5
    bone morphogenic protein-6
    bone morphogenic protein-7
  connective tissue growth factor (CTGF)
  extracellular matrix component
    collagen
    fibrin
    fibrinogen
    fibronectin
  inflammatory cytokine
    basic fibroblast growth factor (bFGF)
    granulocyte-macrophage colony stimulating factor (GM-CSF)
    growth hormones
    insulin growth factor-1 (IGF-1)
    interleukin-1 (IL-1)
    interleukin-6 (IL-6)
    interleukin-8 (IL-8)
    nerve growth factor (NGF)
    platelet-derived growth factor (PDGF)
    transforming growth factor-β (TGF-β)
    tumor necrosis factor-α (TNF-α)
    vascular endothelial growth factor (VEGF)
  leptin
  polymer
    chitosan
    N-carboxybutylchitosan
    a poly(alkylcyanoacrylate)
    poly(ethylene-co-vinylacetate)
    poly(ethylene terephthalate)
    a polylysine
    polytetrafluoroethylene (PTFE)
    a polyurethane
    RGD protein
  vinyl chloride (including a polymer of vinyl chloride)
growth factor
  autologous growth factor
  bovine derived cytokine
  cartilage derived growth factor (CDGF)
  endothelial cell growth factor (ECGF)
  fibroblast growth factor - acidic (aFGF) and basic (bFGF)
  hepatocyte growth factor (HGF)
  insulin growth factor-1 (IGF-1)
  insulin-like growth factor
  nerve growth factor (NGF) (including recombinant NGF)
  platelet-derived endothelial cell growth factor (PD-ECGF)
  platelet-derived growth factor (PDGF)
  tissue necrosis factor (TNF)
  tissue derived cytokine
  transforming growth factors α (TGF-α) & β (TGF-β)
  tumor necrosis factor α (TNF-α)
  vascular endothelial growth factor (VEGF)
  and/or vascular permeability factor (VPF)
hormone
  erythropoietin
nitric oxide or a nitric oxide donor
nitroprusside
nucleic acid
  DNA
  RNA
  siRNA
  microRNA
  antisense
phosphodiesterase inhibitor
prostaglandin inhibitor
proteoglycan
  perlecan
radioactive material
  iodine-125
  iodine-131
  iridium-192
  palladium-103
serotonin blocker
super oxide dismutase
super oxide dismutase mimetic
suramin
thioprotease inhibitor
triazolopyrimidine
tyrosine kinase inhibitor
  ST638
  tyrphostin 9 (AG-17)
vasodilator
  forskolin
  histamine
  nitroglycerin
vitamin
  vitamin C
  1-α-25 dihydroxyvitamin D₃
  vitamin E
```

A fibrosis-inducing agent may include, according to some embodiments, an adhesive, an arterial vessel wall irritant, bleomycin, a bone morphogenic protein (BMP), connective tissue growth factor (CTGF), an extracellular matrix component, an inflammatory cytokine, leptin, a polymer, and/or vinyl chloride (including a polymer of vinyl chloride). In some embodiments, a fibrosis-inducing agent may include analogues and/or derivatives of the foregoing compounds. An adhesive may include, for example, crosslinked poly(ethylene glycol)-methylated collagen and/or cyanoacrylates. An arterial vessel wall irritant may include, for example, crystalline silicates, copper, ethanol, metallic beryllium and oxides thereof, neomycin, quartz dust, silica, silk, talc, talcum powder, and/or wool. A bone morphogenic protein (BMP) may include, for example, bone morphogenic protein-2, bone morphogenic protein-3, bone morphogenic protein-4, bone morphogenic protein-5, bone morphogenic protein-6, and/or bone morphogenic protein-7. An extracellular matrix component may include, for example, collagen, fibrin, fibrinogen, and/or fibronectin. An inflammatory cytokine may include, for example, basic fibroblast growth factor (bFGF), granulocyte-macrophage colony stimulating factor (GM-CSF), growth hormones, insulin growth factor-1 (IGF-1), interleukin-1 (IL-1), interleukin-6 (IL-6), interleukin-8 (IL-8), nerve growth factor (NGF) platelet-derived growth factor (PDGF), transforming growth factor-β (TGF-β), tumor necrosis factor α (TNF-α), and/or vascular endothelial growth factor (VEGF). A polymer may include, for example, chitosan, N-carboxybutylchitosan, a poly(alkylcyanoacrylate), poly(ethylene-co-vinylacetate), poly(ethylene terephthalate), a polylysine, polytetrafluoroethylene (PTFE), a polyurethane, and/or an ROD protein.

A pharmaceutical agent, in some embodiments, may include any compound, mixture of compounds, or composition of matter consisting of a compound, which produces a therapeutic or useful result in at least one subject. A pharmaceutical agent may include a polymer, a marker; such as a radiopaque dye or particles, or may include a drug, including pharmaceutical and therapeutic agents, or an agent including inorganic or organic drugs without limitation. According to some embodiments, a pharmaceutical agent may be in various forms such as an uncharged molecule, a component of a molecular complex, and/or a pharmacologically acceptable salt (e.g., hydrochloride, hydrobromide, sulfate, laurate, palmitate, phosphate, nitrate, borate, acetate, maleate, tartrate, oleate, and salicylate).

In some embodiments, a water insoluble pharmaceutical agent may be included in a scaffold of the disclosure. In other embodiments, a water-soluble derivative of a water insoluble pharmaceutical agent may be included in a scaffold (e.g., to effectively serve as a solute). Once in a subject's body, a water-soluble derivative of a water insoluble pharmaceutical agent may be converted (e.g., by enzymes, hydrolyzed by body pH, or metabolic processes) to a biologically active form. Additionally, a pharmaceutical agent formulation may include various known forms such as solutions, dispersions, pastes, particles, granules, emulsions, suspensions and powders. The drug or agent may or may not be mixed with polymer or a solvent as desired.

A pharmaceutical agent, in some embodiments, may include a solvent. A solvent may be any single solvent or a combination of solvents. For example, a solvent may include water, aliphatic hydrocarbons, aromatic hydrocarbons, alcohols, ketones, dimethyl sulfoxide, tetrahydrofuran, dihydrofuran, dimethylacetamide, acetates, and/or combinations thereof. According to some embodiments, a solvent is ethanol, A solvent is isobutanol in some embodiments. According to some embodiments, two or more pharmaceutical agents may be dissolved or dispersed in the same solvent. For example, dexamethasone, estradiol, and paclitaxel may be dissolved in isobutanol. Alternatively, dexamethasone, estradiol, and paclitaxel may be dissolved in ethanol. In yet another example, dexamethasone, estradiol, and ABT-578, i.e., the rapamycin analog, 3S,6R,7E,9R,10R,12R,14S,15E,17E,19E,21S,23S,26R,27R,34aS)9,10,12,13,14,21,22,23,24, 25,26,27,32,33,34,34a—Hexadecahydro-9,27-dihydroxy-3-[(1R)-2-[(1S,3R,4R)-3-methoxy-4-tetrazol-1-yl)cyclohexyl]-1-methylethyl]-10,21-dimethoxy-6,8,12,14,20,26-hexamethyl-2-3,27-epoxy-3H-pyrido[2,1-c][1,4]oxaazacyclohentriacontine-1,5,11,28,29(4H,6H,31H)-pentone; 23,27-Epoxy-3H-pyrido[2,1-c]i[1,4]oxaazacyclohentriacontin-e-1,5,11,28,29(4H,,6H,31H)-pentone, may be dissolved together in one solvent (e.g., ethanol or isobutanol).

According to some embodiments of the disclosure, a pharmaceutical agent may be a gene therapy agent. For example, a pharmaceutical agent may include a viral or retroviral vector (e.g., adenovirus) having a therapeutic nucleic acid (e.g., a sense or antisense sequence). A pharmaceutical agent may include, for example, a small interfering RNA (siRNA). A siRNA may include a 21 base pair double stranded RNA and may, for example, reduce production of BMP's (e.g., to prevent spinal fusion) or reduce production of cytokines and/or other proteins (e.g., to reduce inflammation and/or promote healing), A siRNA may be complexed with a transfection agent or carrier.

### Methods For Repairing an Annular Defect

A method of repairing an annular defect in a spine may include placing a scaffold implant (e.g., having at least one tail) on, near, and/or adjacent to the defect according to some embodiments. For example, a practitioner (e.g., a surgeon) may choose peri-annular placement or sub-annular placement. In some embodiments, a placement technique may not rely exclusively on the annular fibers to retain the device. Rather, a practitioner may use positive anchoring in the tissues, as allowed by a patient's anatomy. Anchoring may include anchoring directly to the bone of the vertebral endplates and/or anchoring to posterior elements of the vertebra(e). Thus, a method may include, in some embodiments, contacting the at least one thread with a vertebral body of the spine. For example, a method may include threading a tail through a perforation in a vertebral body of the spine. According to some embodiments, a method may also include contacting one or more additional tails through one or more additional perforations in the same and/or another vertebral body of the spine.

To attach a scaffold to vertebral bodies, a practitioner (e.g., a surgeon) may use a tunneling approach, as persons of ordinary skill in the art who have the benefit of the instant disclosure understand. Tunneling in the posterior vertebral endplate anchors the tails of scaffold 200 (as described below), which in turn, anchors the scaffold over the defect. As noted, the scaffold provides reinforcement, which retains the extruded nucleus material.

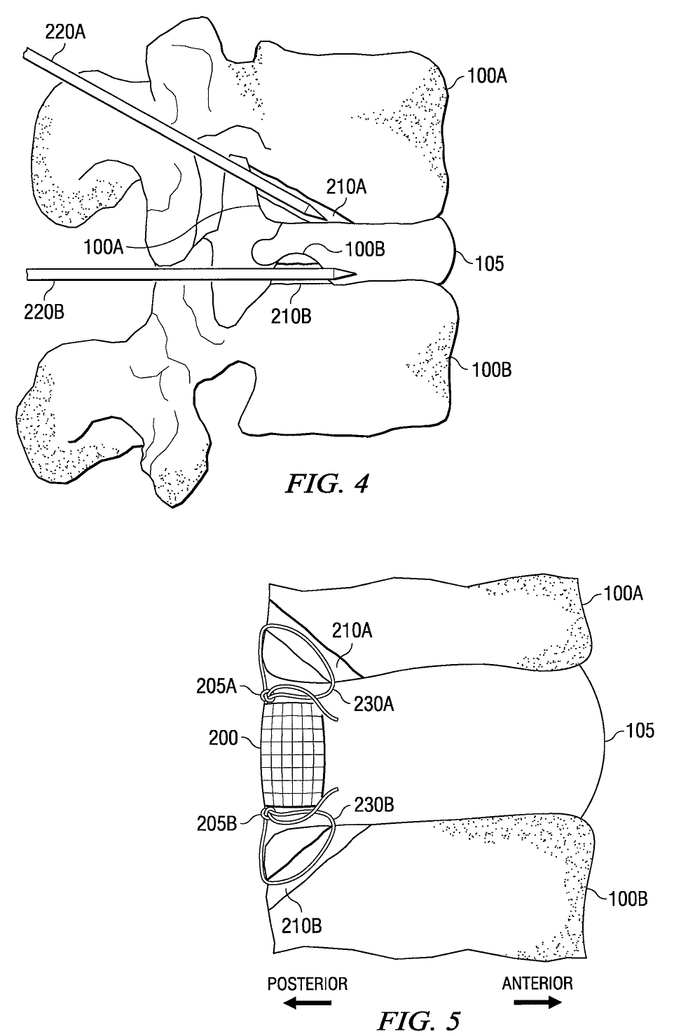

More specifically, a practitioner may make perforation 210A in an endplate of vertebral body 100A. Similarly, a practitioner may make perforation 210B in an endplate of vertebral body 100B. FIG. 4 shows details of making perforation 210A and perforation 210B according to an embodiment of the disclosure. Perforations may be made from a surface substantially normal to the plane of the vertebral endplate, described as a “wall” of the substantially cylindrical surface forming the outer bounds of the anterior vertebral body. A perforation may be made in the wall at an angle or along a curved path with respect to the plane of the endplate, such that a tunnel is created from the wall to the endplate. In one embodiment the tunnel originates on the posterior margin of the vertebral body under the lamina. In another embodiment the tunnel originates in the posterolateral margin of the vertebral body lateral to the facet. Direct lateral or anterior origination points for the tunnel are also possible.

A practitioner may use a variety of techniques and instruments to make perforation 210A and perforation 210B. For example, a practitioner may use a drill, a trochar, or a punch, as desired.

According to the embodiment shown in FIG. 4, a technique may use a pair of trochars to make perforation 210A and perforation 210B. More specifically, a practitioner uses trochar 220A to make perforation 210A in an endplate of vertebral body 100A. A practitioner may make perforation 210A at a desired position, size, and angle (i.e., the angle of penetration of trochar 220A).

Similarly, a practitioner uses trochar 220B to make perforation 210B in an endplate of vertebral body 100B. A practitioner may make perforation 210B at a desired position, size, and angle. If desired, a practitioner may make perforation 210A and perforation 210B at complementary angles with respect to a horizontal (anterior-posterior or top or transverse) plane of annulus 105.

The size, angle, and location of perforation 210A and perforation 210B depend on a variety of factors, as persons of ordinary skill in the art who have the benefit of the instant disclosure understand. The factors include the desired location of scaffold 200 with respect to annulus 105, vertebral body 100A and vertebral body 100B, the patient's anatomy, and the particular geometry and characteristics of scaffold 200 and its tails (as described below).

After performing the perforation procedure above, a practitioner may attach an implant. More specifically, a practitioner may secure one end or region of scaffold 200 to vertebral body 100A by using one or more knots 205A. Likewise, a practitioner may use one or more knots 205B to attach another end of scaffold 200 to vertebral body 100B. As described below in detail, scaffold 200 couples to a pair of tails. A practitioner may use a respective tail to tie knot 205A and knot 205B.

Note that knots constitute just one technique for securing the scaffold implant in a desired location. One may use a variety of techniques to secure the scaffold implant, as persons of ordinary skill in the art who have the benefit of the instant disclosure understand, and as desired. As one example, one may use a crimping tool to crimp a sleeve or other suitable structure in order to secure the implant. As other examples, one may use fraction fits, braids, or cam locks, as desired.

Once attached, an implant may retain the nucleus pulposus, help avoid extrusion of the nucleus pulposus, and/or provide a pharmaceutical (e.g., therapeutic) agent, as described above. An implant may also serve as a scaffold for scar tissue growth, further securing the implant in place.

As noted above, a practitioner may place or implant scaffold 200 in a variety of positions with respect to annulus 105. For example, a practitioner may use a peri-annular placement or an intra-annular placement for scaffold 200 and the implant generally.

FIG. 5 illustrates peri-annular placement of a scaffold implant according to an embodiment of the disclosure. Peri-annular placement refers to placement of scaffold 200 and knots 205A and 205B on or above the surface of annulus 105. Put another way, with peri-annular placement, a practitioner implants scaffold 200 and knots 205A and 20513 superficially with respect to annulus 105.

In some cases of contained herniated nucleus pulposus, peri-annular placement of the scaffold construct reinforces the posterior annulus without accessing the inter-disc space. This method of placement may protect surrounding material from harmful substances contained in the nucleus matter.

Furthermore, some methods of the disclosure may avoid worsening the annular defect, because, for example, a practitioner may place the scaffold on top of the defect. In fact, under some circumstances, a practitioner may even be able to push back the extruded nucleus matter into the defect. In cases where a disc bulge exists, a patch will reinforce the defective area without exposing the body to the nucleus pulposus.

As noted above, scaffold 200 is attached to a pair of tails, shown as tail 230A and tail 230B in FIG. 5. Tail 230A couples or attaches to one end of scaffold 200. Tail 230A couples or attaches to another end of scaffold 200. FIG. 7 and its corresponding discussion provide the topology and construction of the scaffold implant.

Tail 230A and tail 230B allow a practitioner to secure scaffold 200 in a desired location. A practitioner may use tail 230A and tail 230B to tie the implant onto itself. In this manner, a practitioner may avoid using rigid fasteners (e.g., bone screws). Rigid materials may have one or more undesirable effects, such as contact with sensitive nearby tissues or injury to nerves.

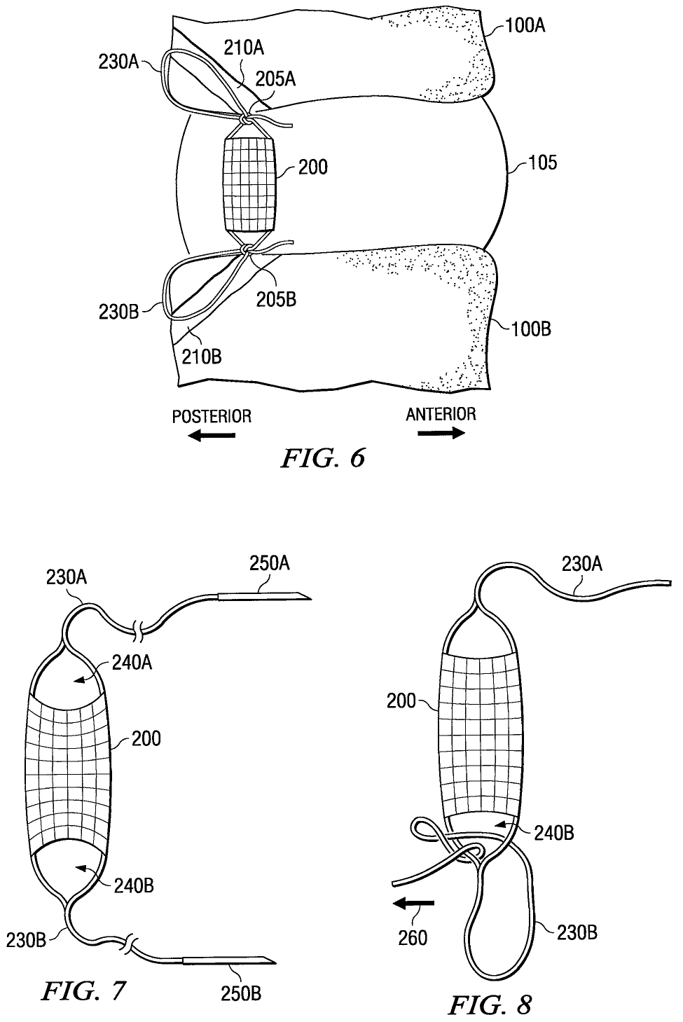

FIG. 6 depicts intra-annular placement of a scaffold implant according to an embodiment of the disclosure. Intra-annular, (or sub-annular or deep) placement of the scaffold implant results in a deeper placement of the implant with respect to annulus 105.

In peri-annular placement, tail 230A and tail 230B enter perforation 210A and 210B, respectively, from the posterior direction of respective vertebral body 100A and vertebral body 100B. In contrast, in intra-annular placement, a practitioner threads tail 230A and 230B so that they enter, respectively, perforation 210A and 210B from near annulus 105 and exit the posterior aspect of vertebral body 100A and vertebral body 100B, respectively.

More specifically, after making perforation 210A, a practitioner may thread tail 230A through perforation 210A, starting with the end of perforation 210A nearer annulus 105. Thus, the free end (i.e., the end not coupled to scaffold 200 before placement of the implant) of tail 230A enters perforation 210A near annulus 105, and exits perforation 210A at the posterior aspect of vertebral body 100A.

After threading through perforation 210A, a practitioner uses the free end of tail 230A to tie knot 205A. A practitioner may pull tail 230A to a desired degree of tension before or during the tying of knot 205A. Once a practitioner has finished tying knot 205A, a practitioner may cut off any excess portion of tail 230A.

Similarly, after making perforation 210B, a practitioner threads tail 230B through perforation 210B. A practitioner begins the threading from an end of perforation 210A that is closer to annulus 105. Thus, the free (i.e., the end not coupled to scaffold 200 before placement of the implant) end of tail 230B enters perforation 210B near annulus 105. After threading, the end of tail 230B exits perforation 210A at the posterior of vertebral body 100B.

After threading through perforation 210B, a practitioner uses the free end of tail 230B to tie knot 205B. As noted above, a practitioner may pull tail 230B to a desired degree of tension before or during the tying of knot 205B. A practitioner may cut off any excess portion of tail 230B after finishing the tying of knot 205B.

FIG. 7 depicts details of a scaffold implant according to an illustrative embodiment of the disclosure. The scaffold implant includes scaffold 200, tail 230A, and tail 230B. Optionally, the implant may include loop 240A and loop 240B. In addition, an implant may optionally include a needle or guide 250A and needle or guide 250B.

Scaffold 200 may be attached to tail 230A and tail 230B, for example, via loop 240A and loop 240B, respectively, or without them. Optional integral loop 240A and loop 240B facilitate the tying of knot 205A and 205B (see FIGS. 5 and 6), respectively (see FIG. 7 and its respective discussion).

As noted, scaffold 200 may cover a herniated region or area of the disc or annulus 105. Scaffold 200 may be permeable or impermeable, as desired. In some embodiments, scaffold 200 may not need to be impermeable. Because scaffold 200 buttresses and supports the herniated region, it may prevent, or tend to prevent, the leakage and release of nucleus material. Furthermore, the patient's body will scar over during the healing process and thus, help to isolate and contain the nucleus material. Thus, a two-stage process may occur in which a permeable scaffold may act to seal the annulus: (1) the permeable scaffold may buttress the insufficient tissue allowing the body to (2) create an impermeable fibrous scar. This configuration may also provide stability to the level (e.g., where the scaffold is able to resist significant tensile forces).

As noted above, a scaffold implant may optionally include needles or guides 250A and 250B coupled to an end of each respective tail (230A and 230B). Needle 250A and needle 250B facilitate threading respective tail 230A and/or tail 230B, tying respective knot 205A and/or knot 205B, and/or both threading and tying.

Once a practitioner has performed the threading, a practitioner may detach (e.g., cut off or otherwise uncouple) needle 250A before tying knot 205A (see FIGS. 5 and 6). Alternatively, a practitioner may use needle 250 in order to aid in tying knot 205A. After threading through perforation 210A, a practitioner may continue to use needle 250A to tie knot 205A. A practitioner may detach (e.g., cut off or otherwise uncouple) needle 250A after tying knot 205A.

Similarly, once a practitioner has threaded tail 230B, a practitioner may detach (e.g., cut off or otherwise uncouple) needle 250B before tying knot 205B (see FIGS. 5 and 6). Alternatively, to facilitate tying, after threading through perforation 210B, a practitioner may continue to use needle 250B to facilitate tying knot 205B. A practitioner may detach (e.g., cut off or otherwise uncouple) needle 250B after tying knot 205B.

One may tie knots 205A and 205B in a variety of ways, as persons of ordinary skill in the art who have the benefit of the instant disclosure understand. As one example, FIG. 8 depicts a technique for tying a knot in a scaffold implant according to an embodiment of the disclosure.

To tie the knot, a practitioner threads the free end of tail 230B through loop 240B in the direction of arrow 260. After the first threading operation, a practitioner then may thread the end of tail 230B one or more times through loop 240B in order to produce a tighter or more secure knot. After the last threading, a practitioner may tie the free end of tail 230B using a conventional knot or surgical knot, as desired.

One may thread tails 230A and 230B through perforations or openings 210A and 210B, respectively, by using a manual approach, or by using an instrument-assisted approach.

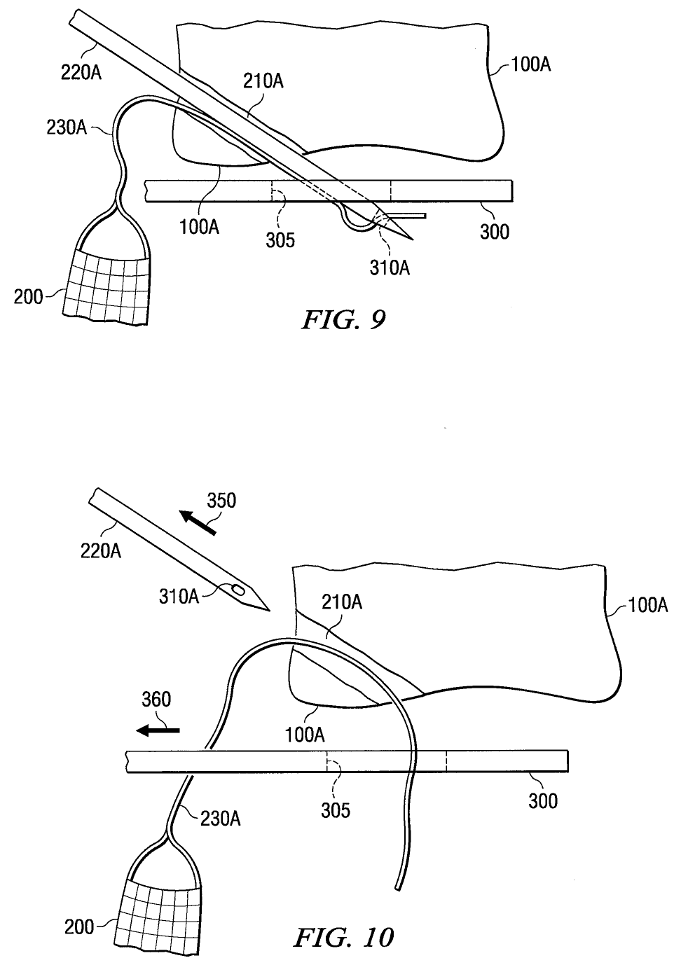

FIGS. 9 and 10 illustrate a manual technique of threading the tails 230A and 230B of the scaffold implant.

In the technique illustrated, a practitioner uses trochar 220 and a plate or guide 300. Trochar 220A has an opening or hole 310A. Likewise, plate 300 has an opening or hole 305. Openings 310A and 305 facilitate the threading of tail 230A. Tail 230B of the scaffold implant is similarly threaded FIGS. 9 and 10 illustrate the threading of tail 230A through perforation 210A of vertebral body 100A. One may use a similar procedure to thread tail 230B through perforation 210B of vertebral body 100B, as persons of ordinary skill in the art who have the benefit of the instant disclosure understand. Optionally, an adhesive may be used to secure a scaffold tail in addition to or in lieu of a knot.

Referring to FIG. 9, a practitioner first threads tail 230A through opening 310A of trochar 220A. A practitioner then inserts trochar 220A into perforation 210A and into opening 305 of plate 300. As trochar 220A travels through perforation 210A of vertebral body 100A, it pulls or carries tail 230A through perforation 210A.

FIG. 10 illustrates how a practitioner completes the threading operation. Once trochar 220A and tail 230A are in their appropriate positions (through opening 305 of plate 300), a practitioner withdraws trochar 220A. A practitioner pulls trochar 220A in the direction generally shown by arrow 350, leaving the free end of tail 230A in opening 305 of plate 300.

Subsequently, a practitioner withdraws plate 300 from the patient's body, using a motion generally in the direction of arrow 360. As plate 300 moves in the direction shown by arrow 360, it pulls or withdraws tie fee end of tail 230A from the patient's body. Once a practitioner has sufficiently withdrawn plate 300, he or she will have access to the free end of tail 230A. A practitioner may then use the retrieved free end of tail 230A to tie a knot and thus secure one end of scaffold 200 in a desired location.

A practitioner may repeat the above technique for the other tail, i.e., tail 230B. Once a practitioner has retrieved tail 230B, he or she may tie another knot, thus securing the second end of scaffold 200 in a desired location. At the conclusion of this procedure, scaffold 200 may be positioned in a desired location with respect to the defect in annulus 105. As one alternative, a practitioner may thread both tail 230A and tail 230B through perforation 305 and retract both tails in direction 360 to secure them.

A method of repairing an annular defect in a spine may include, according to some embodiments, placing a scaffold implant (e.g., having at least one tail) on, near, and/or adjacent to the defect and irradiating the tissue adjacent to the scaffold implant. Irradiation may include ionizing radiation (e.g., beta particles, neutrons, alpha particles, X-rays and photons) and/or proton beams. Gratings, lenses and/or filters may be used to deliver the radiation to a specific site of interest. As one of ordinary skill will understand, the dosing and frequency of irradiation may be adjusted to customize the formation of fibrotic tissue to a particular subject and/or a specific application.

### Methods for Preparing a Spinal Implant

A method of preparing a spinal implant having a scaffold and at least one tail may include, according to some embodiments, providing a scaffold having a bare surface; mixing at least one pharmaceutical agent and at least one polymer in a solvent to form a mixture; and applying the mixture to at least a portion of the bare surface of the scaffold to form a coating thereon. A mixture, in some embodiments, may be applied to the bare surface of the scaffold by spraying, dipping, jetting and/or any other application techniques. According to some embodiments, at least one polymer may be a crosslinkable polymer (e.g., phosphorylcholine-linked methacrylate polymer). The at least one polymer may include a trimethoxysilane functional group in some embodiments. The at least one polymer and at least one pharmaceutical agent may be mixed using ethanol as the solvent. A mixture may be uniformly applied to at least a portion of the scaffold. Also, the at least one pharmaceutical agent may be uniformly distributed in the coating, layered or otherwise disbursed or dissolved in or on the coating or coatings. A coating may have a thickness of about 5 to about 6 microns.

A method, according to some embodiments, may include curing a coating. Curing a coating may include heating the coating, either independently or by way of another processing step in the overall manufacture of a product. Also, a base coating may not be necessary. In some embodiments, a method may further include applying an overcoating to at least a portion of the scaffold.

A coated scaffold may be mounted to a delivery device and/or sterilized, in some embodiments. Sterilization of a coated scaffold may include irradiating the coating. Prior to being sterilized, a coated scaffold may be cured, dried, and/or otherwise processed in accordance with a desired end product. According to some embodiments, a sterilizing step may facilitate crosslinking of the polymer coating. A sterilizing step may include exposing a coated scaffold to at least one cycle of ethylene oxide and/or beat.

A coated scaffold, in some embodiments, may include at least one pharmaceutical agent. For example, a coated scaffold may include about 10 to about 13 micrograms of a pharmaceutical agent along a linear millimeter of the coated scaffold length or as needed to obtain an effective tissue concentration for the required length of time, for the desired end product.

Any dose that leads to a desired or required effective tissue concentration may be used in some embodiments. Effective tissue concentration limits may be known for many drugs. In some such cases, it may be possible to predict the effective tissue concentration when the drug is release from a device. In others, routine dosing experiments may be performed to determine the right dose or desired dose. Concentration of a drug in the tissue may vary with distance from the device and/or may vary in relation to fluid dynamics near the device, e.g., (lymphatic) drainage.

In some embodiments, a scaffold of the present disclosure may include a pharmaceutical agent in any amount desired by a practitioner. One of ordinary skill in the art having the benefit of the present disclosure understands that the exact selection and dose of a pharmaceutical depends on a variety of factors including without limitation, one or more aspects of a subject's medical history (e.g., health, allergies, weight), the intended location of the scaffold, the condition being treated, and the intended course of therapy. A scaffold may include a certain weight of pharmaceutically active agent per unit surface area of device placed in contact with the tissue of interest in order to obtain an effective tissue concentration for the required time. For example, a scaffold may include from about 0.01 micrograms to about 10 milligrams of a pharmaceutical agent along a linear millimeter of the coated scaffold length. For example, a scaffold may include from about 0.01 micrograms to about 0.1 micrograms, from about 0.1 micrograms to about 1.0 micrograms, from about 1.0 micrograms to about 10 micrograms, from about 10 micrograms to about 100 micrograms, from about 100 micrograms to about 1.0 milligram, and/or from about 1.0 milligram to about 10 milligrams of a pharmaceutical agent along a linear millimeter of the coated scaffold length. In some embodiments, these ranges may apply to a scaffold that includes a pharmaceutical agent in its fibers (e.g., rather than as a coating) and/or in domains.

A coated scaffold may include 30% by weight of a therapeutic agent relative to the polymer or as needed for the desired end product. A scaffold, according to some embodiments, may include a pharmaceutical agent in any amount relative to the weight of the scaffold desired by a practitioner. For example, a scaffold may include from about 0.01% by weight to about 0.1% by weight, from about 0.1% by weight to about 1.0% by weight, from about 1.0% by weight to about 10% by weight, from about 1.0% by weight to about 10% by weight, from about 10% by weight to about 20% by weight, from about 20% by weight to about 30% by weight, from about 30% by weight to about 40% by weight, from about 40% by weight to about 50% by weight, and/or more than about 50% by weight of a pharmaceutical agent.

A coating, in some embodiments, may include a uniform matrix of therapeutic agent and polymer; binder, and/or carrier.

One may fabricate scaffold 200, tail 230A and tail 230B, and optional loop 240A and optional loop 240B from a variety of materials, as desired, and as persons of ordinary skill in the art who have the benefit of the instant disclosure understand. The choice of material depends on the desired characteristics of those components, and the particular desired properties of the resulting implant.

Scaffold 200 (and tails 230A and 230B and loops 240A and 240B, as desired) may be fabricated from a natural or synthetic pliable material. The material should be biocompatible and relatively pliable, although one may use a relatively rigid or semi-rigid material, as desired. Furthermore, the materials should encourage fibrous tissue encapsulation.

As an example of one material, one may use polyester to take advantage of its property of encouraging fibrous tissue encapsulation. Various methods are known to persons of ordinary skill in the art for using polyester to encourage tissue in growth. As a specific example, one may use Dacron. One may also coat (e.g., dry coat), impregnate, or micro-texture (or otherwise include or embed into), the material, for example, with therapeutic or medicated agents, to elicit a desired response.

Examples of other materials or therapeutic or medicated agents that may be used include anti-inflammatory agents, anti-adhesive agents (to eliminate or reduce scar tissue), and/or pro-adhesive agents. Examples of anti-inflammatory agents are described in detail in U.S. patent application Ser. No. 11/455,401, titled “Improved Method of Treating Degenerative Spinal Disorders”, filed on Jun. 19, 2006, and incorporated herein by reference). Note, however, that in addition or instead one may use other suitable materials, as persons of ordinary skill in the art who have the benefit of the instant disclosure understand. Furthermore, one may use a single material or agent or a combination of several materials or agents, as desired.

### Systems

A system, according to some embodiments, may include a scaffold implant, together with a tool and/or instrument for positioning or implanting the scaffold implant within a subject's spine. In some embodiments, a tool and/or instrument for positioning or implanting the scaffold implant within a subject's spine may include, for example, a first handle having a channel, a body having a channel, a hollow shaft or tube that connects the channel of the first handle to the channel of the body to form an inserter track, an elongate inserter slidably contained in the inserter track, wherein the inserter has a body end proximal to the body and a first handle end proximal to the first handle, and wherein the body end comprises an opening configured and arranged to receive a spinal implant tail, a second handle attached to the inserter at its first handle end and operable to slide the inserter back and forth along the inserter track, and a pair of articulating needles or guides configured and arranged to contact a spinal implant tail and thread it through a perforation in a vertebral body.

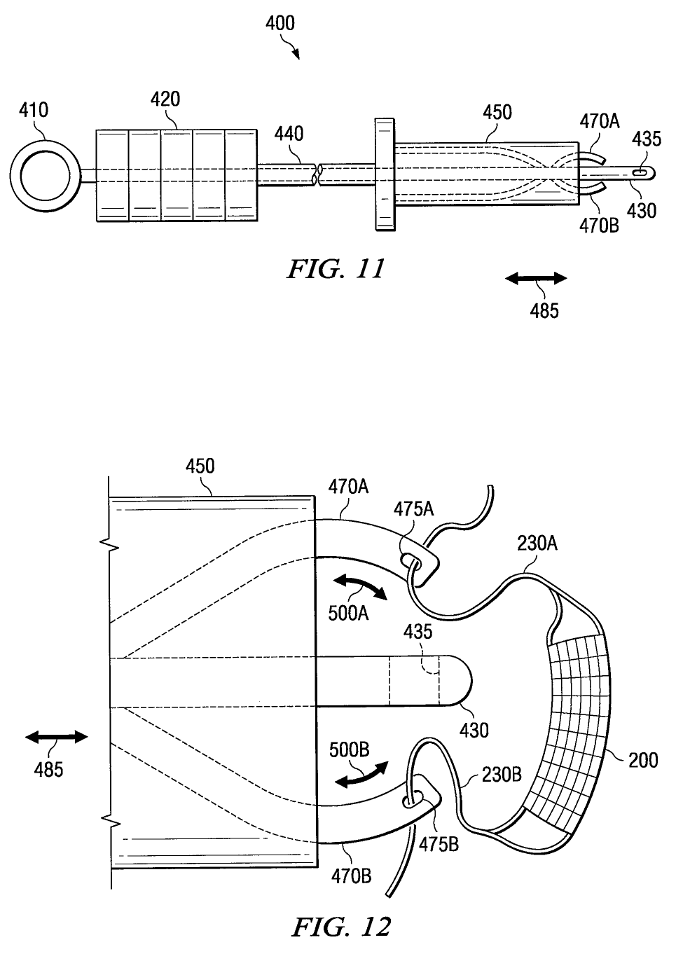

An apparatus, in some embodiments, may include a plate configured to slide within the body, and at least one member configured to thread at least one tail of the scaffold implant. For example, FIG. 11 illustrates an instrument 400 for threading the tails of the scaffold implant into the respective perforations or openings in the spine's vertebral bodies. Instrument 400 includes handle 420, body 450, hollow shaft or tube 440, plate or guide or inserter 430, handle 410 (for plate 430), and a pair of needles or guides 470A and 470B.

Handle 420 provides a mechanism for a practitioner to hold and manipulate instrument 400. Handle 420 couples to shaft 440. Shaft 440 in turn couples to body 450. Thus, handle 420, shaft 440, and body 450 provide a channel through which plate 430 may slide back and forth.

Handle 410 couples to plate 430. Plate 430 may slide through handle 420 of the instrument, through shaft 440, and through body 450. Plate 430 has an opening 435. Tail 230A or tail 230B of the scaffold implant may pass through opening 435.

Handle 410 provides a way for a practitioner to manipulate plate 430. By pushing in or pulling out handle 410, a practitioner may slide plate 430 through body 450. Pushing in handle 410 causes the end of plate 430 to protrude from body 450. Pulling out handle 410 causes the end of plate 430 to retract into body 450.

Needles 470A and 470B provide a mechanism for threading tails 230A and 230B (not shown in FIG. 11) through perforations 210A and 210B (not shown in FIG. 11) of vertebral bodies 100A and 100B (not shown in FIG. 11), respectively. Each of needles 470A and 470B has an opening (see FIG. 12) that allows a respective one of tails 230A and 230B to pass through it.

FIG. 12 depicts details of the operation of the instrument shown in FIG. 11. Plate 430 may slide in or out of body 450 along the direction indicated by arrow 485. Similarly, needles 470A and 470B may move along the directions indicated by arrows 500A and 500B, respectively. In one embodiment, needles 470A and 470B are made from nickel titanium to facilitate actuation along a curved path.

Note that FIG. 12 shows needles 470A and 470B each having an opening (labeled 475A and 475B, respectively). To use instrument 400, a practitioner threads tail 230A through opening 475A of needle 470A. Likewise, a practitioner threads tail 230B through opening 475B of needle 470B.

A practitioner also retracts plate 430 into body 450. A practitioner then inserts needle 470A (along with tail 230A) into perforation 210A (not shown explicitly) of vertebral body 100A (not shown explicitly) by pushing in body 450 in a posterior-to-anterior direction. Similarly, a practitioner inserts needle 470B (along with tail 230B) into perforation 210B (not shown explicitly) of vertebral body 100B (not shown explicitly).

Subsequently, a practitioner slides plate 430 in a posterior-to-anterior direction such that opening 435 of plate 430 becomes aligned or approximately aligned with openings 475A and 475B of needles 470A and 470B, respectively. By pushing needles 470A and 470B through, respectively, perforations 210A and 210B (not shown explicitly), a practitioner causes the threading of tails 230A and 230B through opening 435 of plate 430.

Once tails 230A and 230B thread through opening 435, a practitioner retracts needles 470A and 470B by pulling body 450 in an anterior-to-posterior direction. Needles 470A and 470B consequently retract from perforations 210A and 210B, leaving tails 230A and 230B threaded in opening 435 of plate 430.

A practitioner may then pull handle 410 (not shown in FIG. 12) in an anterior-to-posterior direction in order to retract plate 430 from the patient's body. As plate 430 retracts, it retrieves tails 230A and 230B of the scaffold implant. A practitioner may then secure the scaffold implant in its desired location, using a suitable technique, as described above in detail.

As will be understood by those skilled in the art who have the benefit of the instant disclosure, other equivalent or alternative devices, systems, and methods for spinal implantation at or near an injured and/or damaged annulus fibrosis can be envisioned without departing from the essential characteristics thereof. Accordingly, the manner of carrying out the disclosure as shown and described are to be construed as illustrative only.

Persons skilled in the art may make various changes in the shape, size, number, and/or arrangement of parts without departing from the scope of the instant disclosure. For example, a scaffold may have any regular or irregular curvilinear shape (e.g., triangle, rectangle, square, or other polygon, a circle, an oval, or an ellipse). Also, where ranges have been provides, the disclosed endpoints may be treated as exact and/or estimates as desired or demanded by the particular embodiment. In addition, it may be desirable in some embodiments to mix and match range endpoints. Tail ends may or may not be joined with each other (e.g., using a knot) and/or may be adhered to the bone (tunnel end) using an adhesive material. A pharmaceutical agent may be deposited on a scaffold by any available method. For example, a pharmaceutical agent may be coated (e.g., sprayed or spray-dried) onto a scaffold. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

What is claimed is:

1. A spinal implant, said implant comprising:
a scaffold having a substantially planar surface; and
a plurality of tails, each of which has a first end that is attached to the scaffold and a second end that is configured and arranged to be threaded through a respective perforation in a vertical body,
wherein the scaffold comprises a pharmaceutically effective amount of a pharmaceutical agent.

2. A spinal implant according to claim 1, further comprising a pharmaceutical agent elution matrix comprising the pharmaceutical agent and configured and arranged to release the pharmaceutical agent upon implantation.

3. A spinal implant according to claim 2, further comprising a coating on at least a potion of the spinal implant, the coating comprising the pharmaceutical agent elution matrix.

4. A spinal implant according to claim 2, wherein the scaffold comprises the pharmaceutical agent elution matrix.

5. A spinal implant according to claim 2, wherein the scaffold has a first surface and a second surface.

6. A spinal implant according to claim 5, wherein the first scaffold surface is configured and arranged to face the annulus and nucleus pulposus upon implantation and comprises the pharmaceutical agent elution matrix.

7. A spinal implant according to claim 1, wherein the pharmaceutical agent is selected from the group consisting of an analgesic, an antimicrobial agent, an anti-inflammatory agent, a fibrosis-inducing agent, and combinations thereof.

8. A spinal implant according to claim 1, wherein the pharmaceutical agent comprises a fibrosis-inducing agent selected from the group consisting of an adhesive, an arterial vessel wall irritant, a bone morphogenic protein, an extracellular matrix component, an inflammatory cytokine, a polymer, and combinations thereof.

9. A spinal implant according to claim 1, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

10. A spinal implant according to claim 1, wherein the scaffold comprises a biocompatible material.

11. A spinal implant according to claim 1, wherein the scaffold comprises a biodegradable material.

12. A spinal implant according to claim 1, further comprising a first tail configured and arranged to be threaded through a first perforation in a first vertical body and a second tail configured and arranged to be threaded through a first perforation in a second vertical body.

13. A spinal implant according to claim 12, further comprising a third tail configured and arranged to be threaded through a second perforation in a first vertical body and a fourth tail configured and arranged to be threaded through a second perforation in a second vertical body.

14. A spinal implant according to claim 1, wherein the scaffold comprises polyester, polytetrafluoroethylene, or polyester and polytetrafluoroethylene.

15. A spinal implant according to claim 1, wherein the scaffold comprises a polymer selected from the group consisting of a phosphorylcholine linked macromolecule, an oligoethylenimine, and a polyethylenimine.

16. A spinal implant according to claim 1, wherein the pharmaceutical agent comprises a nucleic acid.

17. A spinal implant, said implant comprising:
an implantable obturator configured and arranged to cover an annular defect, wherein the implantable obturator comprises a pharmaceutical agent; and
a plurality of tails, each of which has a first end that is attached to the implantable obturator and a second end that is configured and arranged to be threaded through a respective perforation in a vertical body.

18. A spinal implant according to claim 17, wherein the implantable obturator is contoured to cover the annular defect and comprises a resilient or rigid material.

19. A spinal implant according to claim 18, wherein the annular defect is on an anterior portion of a disc.

20. A spinal implant according to claim 18, wherein the annular defect is on a posterior portion of a disc.

21. A spinal implant according to claim 18, wherein the annular defect is on a lateral portion of a disc.

22. A spinal implant according to claim 17, further comprising a pharmaceutical agent elution matrix comprising the pharmaceutical agent and configured and arranged to release the pharmaceutical agent upon implantation.

23. A spinal implant according to claim 22, further comprising a coating on at least a potion of the spinal implant, the coating comprising the pharmaceutical agent elution matrix.

24. A spinal implant according to claim 22, wherein the implantable obturator comprises the pharmaceutical agent elution matrix.

25. A spinal implant according to claim 22, wherein the implantable obturator has a first surface and a second surface.

26. A spinal implant according to claim 25, wherein the first implantable obturator surface is configured and arranged to face the annulus and nucleus pulposus upon implantation and comprises the pharmaceutical agent elution matrix.

27. A spinal implant according to claim 17, wherein the pharmaceutical agent is selected from the group consisting of an analgesic, an antimicrobial agent, an anti-inflammatory agent, a fibrosis-inducing agent, and combinations thereof.

28. A spinal implant according to claim 17, wherein the pharmaceutical agent comprises a fibrosis-inducing agent selected from the group consisting of an adhesive, an arterial vessel wall irritant, a bone morphogenic protein, an extracellular matrix component, an inflammatory cytokine, a polymer, and combinations thereof.

29. A spinal implant according to claim 17, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

30. A spinal implant according to claim 17, wherein the pharmaceutical agent comprises a nucleic acid.

31. A spinal implant according to claim 17, wherein the implantable obturator comprises a biocompatible material.

32. A spinal implant according to claim 17, wherein the implantable obturator comprises a biodegradable material.

33. A spinal implant according to claim 17, further comprising a first tail configured and arranged to be threaded through a first perforation in a first vertical body and a second tail configured and arranged to be threaded through a first perforation in a second vertical body.

34. A spinal implant according to claim 33, further comprising a third tail configured and arranged to be threaded through a second perforation in a first vertical body and a fourth tail configured and arranged to be threaded through a second perforation in a second vertical body.

35. A spinal implant according to claim 17, wherein the implantable obturator comprises polyester, polytetrafluoroethylene, or polyester and polytetrafluoroethylene.

36. A spinal implant according to claim 17, wherein the implantable obturator comprises a polymer selected from the group consisting of a phosphorylcholine linked macromolecule, an oligoethylenimine, and a polyethylenimine.

37. A spinal implant according to claim 17, wherein the implantable obturator has a regular curvilinear shape.

38. A spinal implant according to claim 37, wherein the regular curvilinear shape is selected from the group consisting of an oval, a rectangle, a square, and an ellipse.

39. A spinal implant according to claim 37, wherein the implantable obturator is from about 2 mm to about 30 mm along its longest axis.

40. A spinal implant according to claim 37, wherein the implantable obturator is from about 2 mm to about 30 mm along its shortest axis.

41. A spinal implant according to claim 37, wherein the implantable obturator is from about 1 um to about 10 mm at its point of maximum thickness.

42. A system for implanting a spinal implant, said system comprising:
a spinal implant comprising a scaffold having a releasable pharmaceutical agent and a plurality of tails, wherein each tail is configured and arranged to be threaded through a respective perforation in a vertical body; and
an apparatus for placing the spinal implant in or along the spine comprising:
a first handle having a channel, a body having a channel, a hollow shaft or tube that connects the channel of the first handle to the channel of the body to form an inserter track, an elongate inserter slidably contained in the inserter track, wherein the inserter has a body end proximal to the body and a first handle end proximal to the first handle, and wherein the body end comprises an opening configured and arranged to receive at least one of the plurality of tails, a second handle attached to the inserter at its first handle end and operable to slide the inserter back and forth along the inserter track, and a pair of articulating needles or guides configured and arranged to contact at least one of the plurality of tails and thread it through the respective perforation in a vertebral body.

43. A system according to claim 42, wherein the scaffold further comprises a pharmaceutical agent elution matrix comprising the pharmaceutical agent and configured and arranged to release the pharmaceutical agent upon implantation.

44. A spinal implant according to claim 42, wherein the pharmaceutical agent is selected from the group consisting of an analgesic, an antimicrobial agent, an anti-inflammatory agent, a fibrosis-inducing agent, and combinations thereof.

45. A spinal implant according to claim 42, wherein the pharmaceutical agent comprises a fibrosis-inducing agent selected from the group consisting of an adhesive, an arterial vessel wall irritant, a bone morphogenic protein, an extracellular matrix component, an inflammatory cytokine, a polymer, and combinations thereof.

46. A spinal implant according to claim 42, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

47. A system for implanting a spinal implant according to claim 42, wherein at least a portion of the spinal implant system is configured and arranged to be disposable.

48. A method of obturating an annular defect, said method comprising:
contacting the annular defect with a spinal implant comprising
an implantable obturator configured and arranged to cover an annular defect, wherein the implantable obturator comprises a pharmaceutical agent; and
a plurality of tails, each of which has a first end that is attached to the implantable obturator and a second end that is configured and arranged to be threaded through a respective perforation in a vertical body.

49. A spinal implant according to claim 48, wherein the pharmaceutical agent comprises a fibrosis-inducing agent selected from the group consisting of an adhesive, an arterial vessel wall irritant, a bone morphogenic protein, an extracellular matrix component, an inflammatory cytokine, a polymer; and combinations thereof.

50. A spinal implant according to claim 48, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

51. A method of manufacturing a spinal implant, said method comprising:
providing a spinal implant comprising an implantable obturator configured and arranged to cover an annular defect and having a first surface and a second surface, and a plurality of tails, each of which has a first end that is attached to the implantable obturator and a second end that is configured and arranged to be threaded through a respective perforation in a vertical body;
coating the first surface with a pharmaceutical agent elution matrix comprising a pharmaceutical agent; and
sterilizing the spinal implant.

52. A method according to claim 51, wherein the pharmaceutical agent comprises a fibrosis-inducing agent selected from the group consisting of an adhesive, an arterial vessel wall irritant, a bone morphogenic protein, an extracellular matrix component, an inflammatory cytokine, a polymer, and combinations thereof.

53. A method according to claim 51, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylechitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

54. A method according to claim 51, wherein the pharmaceutical agent elution matrix further comprises a polymer selected from the group consisting of a phosphorylcholine linked macromolecule, an oligoethylenimine, and a polyethylenimine.

55. A method of inducing fibrosis at or near an annular defect, the method comprising:
contacting the annular defect with a spinal implant comprising
an implantable obturator configured and arranged to cover an annular defect, wherein the implantable obturator comprises a pharmaceutical agent; and
a plurality of tails, each of which has a first end that is attached to the implantable obturator and a second end that is configured and arranged to be threaded through a respective perforation in a vertical body.

56. A method according to claim 55, wherein the pharmaceutical agent comprises a fibrosis-inducing agent selected from the group consisting of an adhesive, an arterial vessel wall irritant, a bone morphiogenic protein, an extracellular matrix component, an inflammatory cytokine, a polymer, and combinations thereof.

57. A method according to claim 55, wherein the pharmaceutical agent comprises a fibrosis-inducing agent selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof.

58. A method according to claim 55, wherein the pharmaceutical agent comprises a nucleic acid.

59. A method according to claim 55, further comprising irradiating the annular defect.
